(19) United States
(12) Patent Application Publication
(10) Pub. No.: US 2025/0127754 A1
(43) Pub. Date: Apr. 24, 2025

(54) Pharmaceutical Application of Dimyricetin-yl-diselenide in Atherosclerosis

(71) Applicant: Shanghai Spark Pharmaceutical Co., Ltd., Shanghai (CN)

(72) Inventors: Kunyuan SONG, Shanghai (CN); Weiwei CHEN, Shanghai (CN)

(21) Appl. No.: 18/920,055

(22) Filed: Oct. 18, 2024

(30) Foreign Application Priority Data
Oct. 23, 2023 (CN) 202311377639.7

(51) Int. Cl. A61K 31/352; A61P 9/10

(52) U.S. Cl. CPC A61K 31/352; A61P 9/10

(57) ABSTRACT

This application discloses the use of dimyricetin-yl-diselenide in the preparation of a pharmaceutical composition for preventing, alleviating and/or treating cardiovascular diseases, and for preventing and/or treating atherosclerosis.

### TECHNICAL FIELD

This application relates to the field of pharmaceutical technology, and in particular to the pharmaceutical application of a dimyricetin-yl-diselenide.

### BACKGROUND OF RELATED ARTS

Due to population aging, changes in lifestyle, and the continuous prevalence of metabolic risk factors, the prevalence of cardiovascular diseases (CVDs) has been increasing globally year by year. In China, due to the large population base, the health and economic burden caused by CVDs is particularly severe. In 2019, CVDs accounted for 46.74% and 44.26% of deaths in rural and urban areas, respectively, with 2 out of every 5 deaths attributed to CVDs. Among CVDs, coronary atherosclerotic heart disease (CAD), also known as coronary heart disease, is one of the diseases with the highest morbidity and mortality rates. Although the progressive popularization of interventional therapy and the development of lipid-regulating drugs such as statins have allowed for interventions in conventional high-risk factors of atherosclerosis, including hyperlipidemia, smoking, hypertension, and diabetes, the disease progression in CAD patients has been contained, and the risk of death has also decreased. Nevertheless, CAD remains one of the leading causes of death globally.

Atherosclerosis (AS) is the primary cause of CAD, cerebral infarction, and peripheral vascular diseases. Dyslipidemia serves as the pathological basis of AS, characterized by the initiation of lesions in the intima of affected arteries. Typically, this involves the accumulation of lipids and complex sugars, hemorrhage, and thrombosis, followed by fibrous tissue proliferation, calcium deposition, and gradual degeneration and calcification of the arterial media, leading to thickening and hardening of the arterial wall and stenosis of the vascular lumen. These lesions often affect large and medium-sized muscular arteries. Once the lesions develop to the extent that they obstruct the arterial lumen, the tissues or organs supplied by that artery will suffer from ischemia or necrosis.

Selenium is an essential trace element for humans. The Chinese Nutrition Society has also listed selenium as one of the 15 essential nutrients for humans. Numerous clinical trials conducted both domestically and internationally have demonstrated that selenium deficiency in humans can cause dysfunction of certain vital organs, leading to the occurrence of many serious diseases. Studies have shown that moderate selenium supplementation in selenium-deficient individuals can not only prevent the occurrence of tumors, liver diseases, etc., but also enhance the immune function of the body, maintain the normal function of vital organs such as the heart, liver, lungs, and stomach, and prevent the occurrence of geriatric cardiovascular and cerebrovascular diseases.

Myricetin is a flavonoid compound widely found in the bark and leaves of bayberries, exhibiting various pharmacological activities such as anti-inflammatory, anti-tumor, anti-mutagenic, anti-caries, antioxidant, and free radical scavenging properties.

Diselenides possess significant antioxidant properties and can mimic the activity of glutathione peroxidase (GSH-PX). Some diselenides also exhibit antitumor, antibacterial, bactericidal, and disinfectant activities.

Dimyricetin-yl-diselenide (DMS), with a molecular formula of C30H18016Se2 and a molecular weight of 792.37, is a novel compound discovered by our company. It appears as a pale yellow powder.

### SUMMARY

This application provides a pharmaceutical application of dimyricetin-yl-diselenide.

The technical solution adopted in this application is as follows: The use of dimyricetin-yl-diselenide for preparing pharmaceutical compositions, which are used for preventing, alleviating, and/or treating cardiovascular diseases, as well as preventing and/or treating atherosclerosis.

The beneficial effects of this application are as follows:

Experiments have confirmed that dimyricetin-yl-diselenide is effective in preventing and/or treating atherosclerosis.

### BRIEF DESCRIPTION OF THE DRAWINGS

The figures described herein are intended to provide further understanding of the present application and constitute a part of this application. The illustrative embodiments and descriptions of the present application are used to explain the present application and do not constitute improper limitations to the present application. In the figures:

FIG. 1 shows the effect of the test drug on the body weight of mice.

FIG. 2 demonstrates the impact of the test drug on blood lipids and plasma LDH levels in ApoE−/− mice.

FIG. 3 illustrates the effect of the test drug on plasma SOD and MDA levels in ApoE−/− mice.

FIG. 4 presents the influence of the test drug on aortic plaques in ApoE−/− mice.

FIG. 5 further elaborates on the effect of the test drug on aortic plaques in ApoE−/− mice.

FIG. 6 indicates the influence of the test drug on heart weight index in ApoE−/− mice.

### DETAILED DESCRIPTIONS OF EMBODIMENTS

### Detailed Implementation Mode

To make the objectives, technical solutions, and advantages of this application clearer, the following will provide a clear and comprehensive description of the technical solutions of this application by combining specific embodiments of this application and corresponding drawings. Obviously, the described embodiments are only part of the embodiments of this application, not all of them. All other embodiments obtained by ordinary technicians in the field without creative work based on the embodiments of this application fall within the scope of protection of this application.

### Embodiment: Study on the Pharmacodynamic Effect of Atherosclerosis Model in ApoE−/− Mice with High-Fat Diet

### I. Experimental Animals

### 1.1 Experimental Animals

ApoE−/− mice (ApoE gene knockout mice) aged 56-62 days, male, SPF grade, were provided by Beijing Vital River Laboratory Animal Technology Co., Ltd. License number: SCXK (Beijing) 2021-0006, qualification certificate number: No. 110011230100310621.

### 1.2 Breeding Environment

All animals used in the experiment were ApoE−/− mice. Upon arrival at WuXi AppTec Pharmaceutical Technology (Nantong) Co., Ltd., they were housed in SPF-grade animal rooms with strictly controlled environmental conditions. The temperature in the breeding rooms was maintained at 20-25° C., and the humidity was maintained at 40-70%. Temperature and humidity in the breeding rooms were monitored in real-time using hygrometers, and readings were recorded twice daily (once in the morning and once in the afternoon). Lighting in the animal rooms was controlled by an electronic timer system, with 12 hours of light followed by 12 hours of darkness (lights on at 6:00 AM and off at 18:00 PM). Animals had free access to feed and ensured ad libitum drinking water.

### II. Experimental Methods

### 2.1 Experimental Materials and Instruments

| | | PRODUCTION |
|---|---|---|
| | | LOT |
| REAGENT | SUPPLIER | NUMBER |
| TRIG test kit | Beckman | AUZ 0735 |
| CHOL test kit | Beckman | AUZ 0608 |
| HDL test kit | Beckman | AUZ 1158 |
| LDL test kit | Beckman | AUZ 1232 |
| LDH test kit | Beckman | AUZ 0982 |
| SOD test kit | Beyotime Biotechnology | 030223230412 |
| | Co., LTD | |
| MDA test kit | Beijing Applygen | 2023R2G |
| | Technology Co., LTD | |
| 0.9% Sodium | Huayu (Wuxi) | 20011402 |
| chloride injection | Pharmaceutical Co., LTD | |
| DMSO | RK8RRQ1Q | Anhui Zesheng |
| | | Technology Co., LTD |
| PEG400 | R20120011 | Shanghai Accela |
| | | Reagent Co., Ltd. |
| Glacial acetic | Energy Chemical Technology | DX3REA9K |
| acid | (Shanghai) Co., LTD | |
| Oil-red O | Sigma | SLCG5388 |
| Isopropyl alcohol | Sinopharm Group Chemical | 20210605 |
| | reagent Co., LTD | |
| 4% | Beyotime Biotechnology | 112922230324 |
| Paraformaldehyde | Co., LTD | |

### 2.1.1 Reagents and Suppliers

| | | PRODUCT |
|---|---|---|
| INSTRUMENT | SUPPLIER | MODEL |
| Electronic balance | Changzhou Tianzhiping | YH-2000 |
| | instrument equipment | |
| | Co., LTD | |
| Electronic balance | Sartorius Scientific | SOP |
| | Instruments (Beijing) | |
| | Co., LTD | |
| Small animal ultrasound | Fujifilm visualsonics | Vevo1100 |
| imaging system | | |
| centrifuge | Eppendorf | 5424R |
| Asana mirror | LECIA | MZ6 |
| Constant temperature | Shanghai Sile | 85-2 type |
| magnetic stirrer | Instrument Co., LTD | |
| Multi-functional shift | Hangzhou Qiwei | QW-YC-29ST |
| shaking table | Instrument Co., LTD | |
| Biochemical analyzer | Beckman | AU480 |
| camera | Canon | E0S800D |

### 2.2 Experimental Procedure

Upon arrival, ApoE−/− mice were acclimatized to the experimental facility for 3 days. After an overnight fast (12 hours), blood samples were collected to measure baseline levels of serum lipids (TC, TG, HDL-C, LDL-C) and LDH. Control mice were fed a regular diet, while the remaining mice were fed a high-fat diet for 18 weeks. The regular diet contained 11% kcal fat and 0% cholesterol, while the high-fat diet contained 40% kcal fat and 0.21% cholesterol. At 8 weeks of high-fat feeding, the animals underwent echocardiography and blood biochemical tests. Based on their TC values, they were randomly assigned to the following groups: blank control, model control, positive control, AQ-C-a, DMS-ASD, and DMS (refer to Table 2 in Section 6.5). Drug administration continued for 10 weeks. During the experiment, the animals' appearance, behavior, and physical condition were observed, and their body weight, echocardiography, serum lipids (TC, TG, HDL-C, and LDL-C), and blood biochemistry (LDH, SOD, and MDA) were monitored. At the end of the experiment (Week 18), hearts and aortas were collected for Oil Red O staining and histopathological analysis.

### 2.3 Test Article and High-Fat Diet

| Name | Lot number | SUPPLIER | REMARK |
|---|---|---|---|
| Atorvastatin | A2314019 | Beijing Inno | Positive drug |
| | | Technology Co., LTD | |
| AQ-C-a | 20220829 | Shanghai Spark | DMS Solid |
| | | Pharmaceutical Co., | Preparation 1 |
| | | Ltd. | |
| DMS-ASD | 20230101 | Shanghai Spark | DMS Solid |
| | | Pharmaceutical Co., | Preparation 2 |
| | | Ltd. | |
| DMS | 210701R | Shanghai Spark | DMS Liquid |
| | | Pharmaceutical Co., | preparation |
| | | Ltd. | |
| Conventional | NA | Jiangsu Xietong | / |
| feed | | Pharmaceutical Bio- | |
| | | engineering Co., Ltd. | |
| High fat | 23020109 | Chengdu Aobo | |
| feedD12079B | | Biotechnology Co., LTD | |

### 2.4 Drug Preparation

Solvent 1: 5% DMSO+95% DD water;
Solvent 2: 20% DMSO+60% PEG400+20% 10 mM Acetic buffer (pH 4.5)

1) Weigh an appropriate amount of AQ-C-a and add it to 5% DMSO. Vortex for 1 minute, then dilute with water to the desired concentration. Vortex again for 1 minute and set aside for immediate use. The solution is a suspension and should be prepared fresh before use.

2) Weigh an appropriate amount of DMS-ASD and add it to 5% DMSO. Vortex for 1 minute, then dilute with water to the desired concentration. Vortex again for 1 minute and set aside for immediate use. The solution is a suspension and should be prepared fresh before use.

3) Weigh an appropriate amount of DMS, determine the final volume based on the desired concentration, and add it to 20% DMSO. Vortex for 1 minute, then add 60% PEG400 and vortex for another 1 minute. Subsequently, add 20% 10 mM Acetic buffer (pH 4.5) and vortex for 1 minute. Set aside for immediate use. This solution should be prepared fresh before use.

Storage: The compounds should be prepared on the day of administration. Store at 4° C. first, and take them out of the refrigerator to prepare according to the method before use.

### 2.5 Experimental Grouping

| | | | | Route of | |
|---|---|---|---|---|---|
| | | | dose | adminis- | Sched- |
| Group | Plan | Quantity | (mg/kg) | tration | ule |
| 1 | Regular diet + | 8 | — | P.O. | QD |
| | Vehicle | | | | |
| 2 | HFD + Vehicle | 6 | — | P.O. | QD |
| 3 | HFD + | 6 | 20 | P.O. | QD |
| | Atorvastatin | | | | |
| 4 | HFD + AQ-C-a | 6 | 300 | P.O. | QD |
| 5 | HFD + DMS-ASD | 6 | 300 | P.O. | QD |
| 6 | HFD + DMS | 8 | 20 | SC | QD |

### 2.6 Endpoint Detection

### 2.6.1 Weight

All animals were given medicine once a week, and once a day during the medication period;

### 2.6.2 Biochemical Blood Examination

Animals were fasted the night before blood collection. Blood samples were collected and anticoagulated with heparin to prepare plasma for the detection of TG, TC, HDL-C, LDL-c, and LDH. Blood was collected at six time points: Week 0, Week 8, Week 10, Week 12, Week 16, and Week 18. SOD and MDA levels were measured at Week 16 and Week 18.

### 2.6.3 Aortic Fat Plaque Examination

Aortic oil red O staining was performed. The experimental endpoint was set at Week 10 post-treatment. After euthanasia, the aorta was collected from the aortic arch to the iliac artery and subjected to oil red O staining to analyze the accumulation of fat plaques in the aorta.

### 2.6.4 Heart Weight and Outflow Tract Plaque Assessment

At the experimental endpoint, hearts and tibias were collected. The heart was weighed and its weight index was calculated using tibial length for correction, with the formula: Heart Index=Heart Weight (g)/Tibial Length (cm)×100%. The heart was fixed in 4% PFA and subjected to HE staining to evaluate fat plaques in the outflow tract.

### 2.7 Data Analysis

All data were entered into an Excel document and presented as mean±standard error. Significant differences were compared using one-way ANOVA. A P-value less than 0.05 was considered statistically significant, and a P-value less than 0.01 was considered highly statistically significant.

### III. Results

### 3.1 Effect on Average Body Weight Changes in ApoE−/− Mice

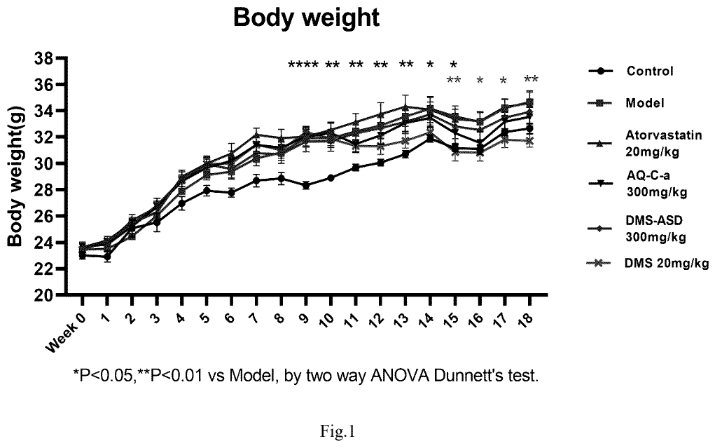

As shown in FIG. 1, ApoE−/− mice fed with HFD exhibited significantly higher average body weight gain compared to control mice. Starting from the 9th week, the body weights of mice in the model, atorvastatin, and test drug AQ-C-a and DMS-ASD groups were all significantly greater than those in the control group (P<0.05), with no significant differences observed among these groups (P>0.05). The body weight gain in the DMS test drug group stabilized from the 10th to 11th week and overlapped with the control group's weight curve by the 14th week.

### 3.2 Effect on Four Lipid Parameters and Plasma LDH Content in ApoE−/− Mice

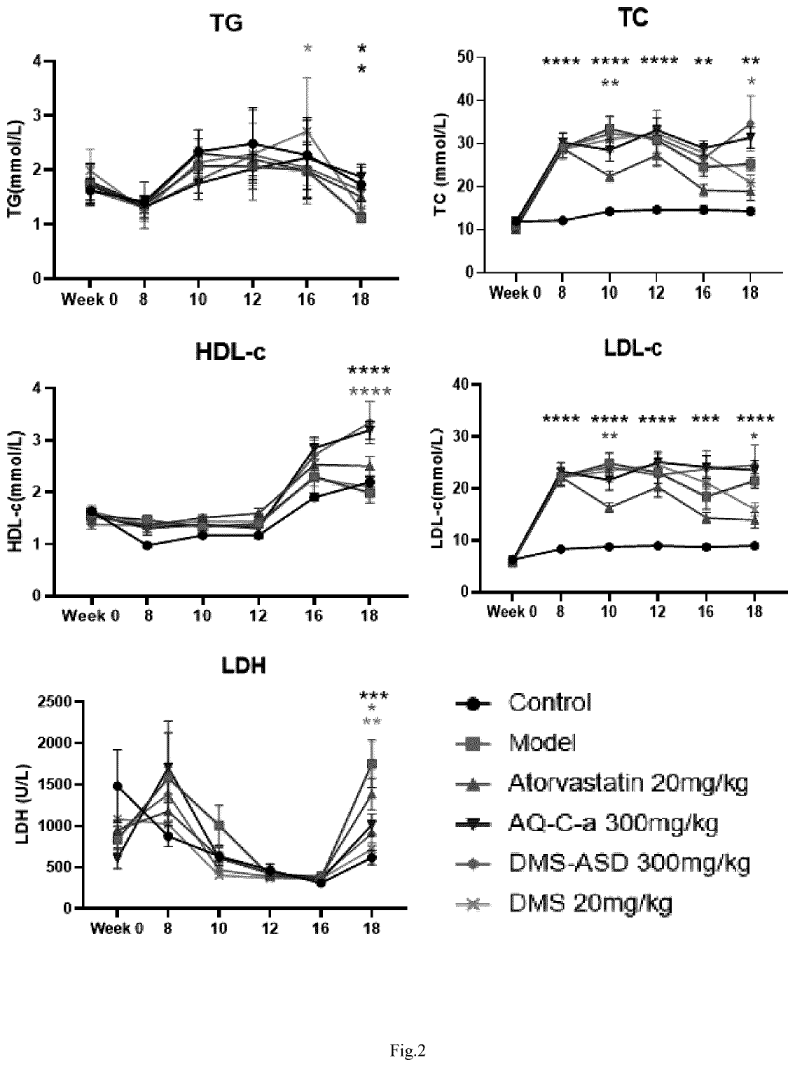

As presented in Table 1 and FIG. 2, long-term HFD feeding led to abnormal elevations in blood lipid levels in mice. Compared to the control group, ApoE−/− mice in the model group showed significant increases in TC and LDL-c after 8 weeks of HFD feeding (P<0.05), while TG and HDL-c remained unchanged (P>0.05). Compared to the model group, atorvastatin significantly reduced TC and LDL-c levels. The test drug DMS showed a trend of reducing TC and LDL-c in ApoE−/− mice from the 16th to 18th week, but this was not statistically significant (P>0.05). Test drugs AQ-C-a and DMS-ASD had no significant effects on TC and LDL-c in ApoE−/− mice (P>0.05), but both significantly increased HDL-c levels at the 18th week (P<0.001).

At the 18th week, plasma LDH levels in model mice were significantly higher than those in the control group. Atorvastatin and all test drug groups, particularly DMS-ASD and DMS mice, exhibited significant reductions in plasma LDH compared to the model group (P<0.05) (Table 1 and FIG. 2).

TABLE 1
Effects of tested drugs on the four blood lipid indicators
and LDH content in ApoE−/− mice

| | Time | | | |
|---|---|---|---|---|
| Group | (week) | Contrast | Model | Atorvastatin |
| Dose | — | — | — | 20 mg/kg |
| TG | 0 | 1.63 ± 0.08 | 1.75 ± 0.15 | 1.79 ± 0.14 |
| | 8 | 1.42 ± 0.04 | 1.35 ± 0.06 | 1.35 ± 0.17 |
| | 10 | 2.34 ± 0.14 | 2.07 ± 0.2 | 2.31 ± 0.18 |
| | 12 | 2.49 ± 0.23 | 2.07 ± 0.17 | 2.2 ± 0.13 |
| | 16 | 2.28 ± 0.23 | 1.99 ± 0.22 | 1.99 ± 0.2 |
| | 18 | 1.74 ± 0.11 | 1.12 ± 0.03 | 1.51 ± 0.09 |
| TC | 0 | 11.95 ± 0.79 | 10.81 ± 0.82 | 10.12 ± 0.84 |
| | 8 | 12.17 ± 0.34 | 28.99 ± 2.17**** | 28.87 ± 2.19**** |
| | 10 | 14.25 ± 0.65 | 33.39 ± 2.92 | 22.38 ± 1.25** |
| | 12 | 14.63 ± 0.64 | 30.77 ± 3.03 | 27.29 ± 2.61 |
| | 16 | 14.66 ± 0.83 | 24.5 ± 2.2 | 19.14 ± 1.34 |
| | 18 | 14.3 ± 0.81 | 25.23 ± 1.53 | 18.89 ± 2.11 |
| HDL-C | 0 | 1.64 ± 0.08 | 1.55 ± 0.14 | 1.53 ± 0.15 |
| | 8 | 0.98 ± 0.05 | 1.46 ± 0.09 | 1.36 ± 0.13 |
| | 10 | 1.17 ± 0.05 | 1.33 ± 0.19 | 1.51 ± 0.06 |
| | 12 | 1.17 ± 0.05 | 1.39 ± 0.18 | 1.59 ± 0.09 |
| | 16 | 1.9 ± 0.07 | 2.29 ± 0.31 | 2.53 ± 0.12 |
| | 18 | 2.2 ± 0.09 | 1.99 ± 0.2 | 2.5 ± 0.18**** |
| LDL-C | 0 | 6.26 ± 0.28 | 5.79 ± 0.15 | 5.63 ± 0.34 |
| | 8 | 8.31 ± 0.25 | 22.18 ± 1.71**** | 22.23 ± 1.6**** |
| | 10 | 8.76 ± 0.37 | 24.83 ± 1.85 | 16.31 ± 0.92** |
| | 12 | 8.96 ± 0.38 | 23.03 ± 2.07 | 20.24 ± 2 |
| | 16 | 8.69 ± 0.5 | 18.39 ± 2.44 | 14.32 ± 1.08 |
| | 18 | 8.94 ± 0.5 | 21.44 ± 1.42 | 13.87 ± 1.51* |
| LDH | 0 | 1483 ± 439.35 | 836 ± 161.58 | 948.67 ± 117.19 |
| | 8 | 877 ± 129.04 | 1580.17 ± 690.06 | 1176.67 ± 142.27 |
| | 10 | 634 ± 108.94 | 1005.33 ± 242.18 | 620 ± 62.03 |
| | 12 | 465.25 ± 75.63 | 392.67 ± 38.05 | 422.67 ± 50.59 |
| | 16 | 309.75 ± 15.04 | 420.83 ± 71.76 | 384 ± 25.93 |
| | 18 | 618.5 ± 90.36**** | 2437.17 ± 726.42 | 1384.67 ± 191.24 |

| | Time | | | |
|---|---|---|---|---|
| Group | (week) | AQ-C-a | DMS-ASD | DMS |
| Dose | — | 300 mg/kg | 300 mg/kg | 20 mg/kg |
| TG | 0 | 1.72 ± 0.14 | 1.64 ± 0.08 | 1.99 ± 0.14 |
| | 8 | 1.32 ± 0.07 | 1.31 ± 0.08 | 1.26 ± 0.07 |
| | 10 | 1.75 ± 0.11 | 1.81 ± 0.14 | 2.14 ± 0.1 |
| | 12 | 2.02 ± 0.1 | 2.28 ± 0.34 | 2.29 ± 0.2 |
| | 16 | 2.23 ± 0.28 | 2.04 ± 0.27 | 2.71 ± 0.35* |
| | 18 | 1.87 ± 0.09 | 1.63 ± 0.2 | 1.27 ± 0.06 |
| TC | 0 | 11.84 ± 1.05 | 10.3 ± 0.82 | 11.84 ± 0.85 |
| | 8 | 30.23 ± 2.24** | 28.96 ± 2.17** | 28.69 ± 2.34** |
| | 10 | 28.47 ± 2.55 | 32.25 ± 4.14 | 30.95 ± 1.95 |
| | 12 | 33.09 ± 2.86 | 31.38 ± 6.36 | 32.35 ± 1.96 |
| | 16 | 28.94 ± 1.69 | 26.57 ± 2.45 | 27.78 ± 1.36 |
| | 18 | 31.36 ± 2.44 | 34.66 ± 6.4 | 20.92 ± 1.78* |
| HDL-C | 0 | 1.57 ± 0.12 | 1.37 ± 0.09 | 1.63 ± 0.12 |
| | 8 | 1.3 ± 0.13 | 1.37 ± 0.05 | 1.38 ± 0.15 |
| | 10 | 1.37 ± 0.11 | 1.35 ± 0.18 | 1.44 ± 0.07 |
| | 12 | 1.30 ± 0.11 | 1.35 ± 0.24 | 1.44 ± 0.07 |
| | 16 | 2.85 ± 0.21 | 2.71 ± 0.29 | 2.27 ± 0.15 |
| | 18 | 3.19 ± 0.18 | 3.34 ± 0.41**** | 2.1 ± 0.19 |
| LDL-C | 0 | 6.09 ± 0.18 | 5.84 ± 0.37 | 6.07 ± 0.19 |
| | 8 | 23.24 ± 1.75**** | 22.28 ± 1.63*** | 22.09 ± 1.86**** |
| | 10 | 21.63 ± 1.94 | 24.1 ± 2.86 | 23.38 ± 1.46 |
| | 12 | 25.06 ± 2.02 | 22.52 ± 4.13 | 23.38 ± 1.46 |
| | 16 | 24.12 ± 2.16 | 23.75 ± 3.58 | 21.1 ± 1.23 |
| | 18 | 23.53 ± 1.84 | 24.51 ± 3.88 | 15.94 ± 1.32 |
| LDH | 0 | 605.71 ± 122.68 | 924.67 ± 143.59 | 1083.25 ± 375.4 |
| | 8 | 1706.86 ± 419.42 | 1382.67 ± 328.35 | 1022.5 ± 112.95 |
| | 10 | 608.57 ± 38.57 | 466 ± 45.95 | 401.5 ± 22.95 |
| | 12 | 427.43 ± 24.42 | 386 ± 23.2 | 368.5 ± 26.82 |
| | 16 | 350.71 ± 48.66 | 386.83 ± 43.07 | 361.88 ± 20.78 |
| | 18 | 1014 ± 133.86 | 904.67 ± 144.76* | 719 ± 81.08** |

### 3.3 Effect on SOD and MDA in Plasma of ApoE−/− Mice

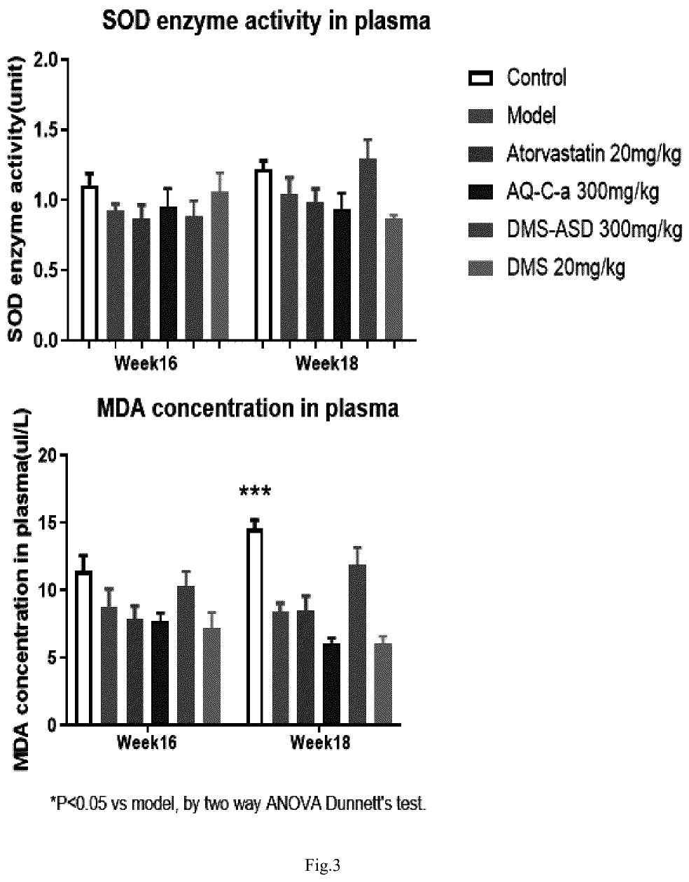

After 8 weeks (week 16) and 10 weeks (week 18) of drug administration, the SOD and MDA levels in the plasma of mice are shown in Table 2 and FIG. 3. There was no significant difference in SOD activity between all groups at week 16 and week 18 (P>0.05). At week 18, the MDA content in the model group was significantly lower than that in the control group (P<0.05), while no statistically significant differences were observed among other groups (P>0.05) (Table 2 and FIG. 3).

TABLE 2
Effects of tested drugs on SOD and MDA in plasma of ApoE−/− mice

| | Time | | | | | | |
|---|---|---|---|---|---|---|---|
| Group | (week) | Contrast | Model | Atorvastatin | AQ-C-a | DMS-ASD | DMS |
| Dose | — | — | — | 20 mg/kg | 300 mg/kg | 300 mg/kg | 20 mg/kg |
| SOD | 16 | 1.11 ± 0.08 | 0.93 ± 0.04 | 0.87 ± 0.09 | 0.95 ± 0.13 | 0.88 ± 0.11 | 1.06 ± 0.14 |
| (U/mL) | 18 | 1.22 ± 0.06 | 1.05 ± 0.11 | 0.99 ± 0.09 | 0.94 ± 0.11 | 1.30 ± 0.14 | 0.87 ± 0.03 |
| MDA | 16 | 11.40 ± 1.15 | 8.74 ± 1.35 | 7.86 ± 0.95 | 7.67 ± 0.63 | 10.29 ± 1.11 | 7.19 ± 1.15 |
| (μL/L) | 18 | 14.54 ± 0.66*** | 8.38 ± 0.64 | 8.64 ± 1.10 | 6.10 ± 0.34 | 11.85 ± 1.31 | 6.08 ± 0.50 |

### 3.4 Effects on the Proportion of Aortic Plaque Area in ApoE−/− Mice

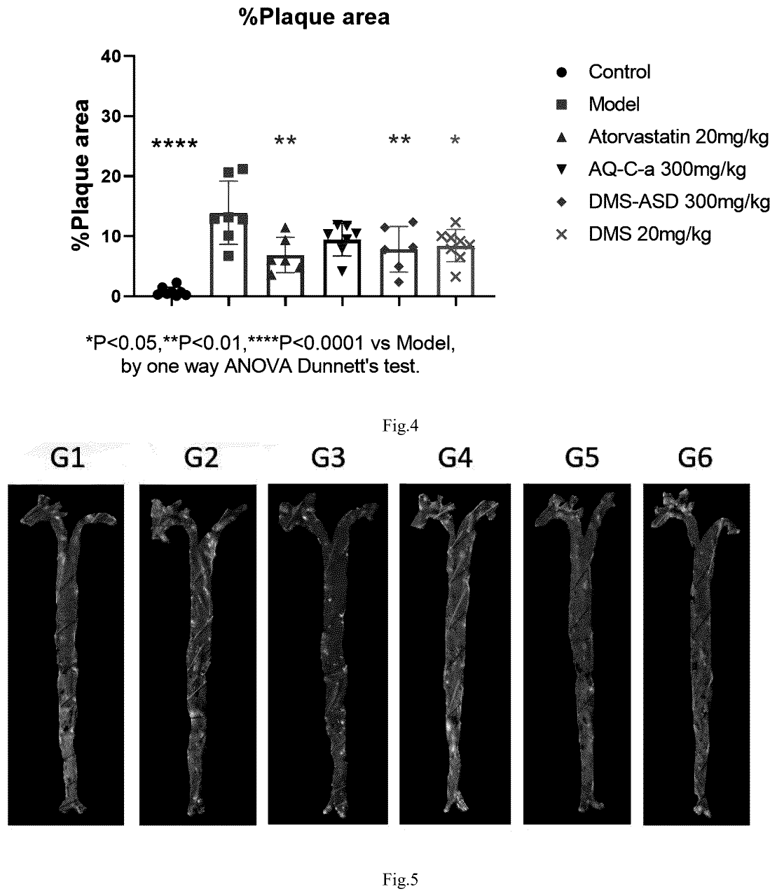

At the end of the 18th week of the experiment, oil red staining and quantitative analysis of atherosclerotic plaque area in the mouse aorta were performed. The results are shown in Table 3, FIG. 4, and FIG. 5: The percentage of aortic plaque in the model group was significantly higher than that in the control group (12.71±1.88 vs 0.77±0.26, P<0.0001). Compared with the model group, the positive control atorvastatin significantly reduced the percentage of aortic plaque caused by HFD (P<0.01); the test drugs AQ-C-a, DMS-ASD, and DMS also showed a reduction in the percentage of aortic atherosclerotic plaque in mice, especially DMS-ASD and DMS, which were statistically significant compared with the model group (P<0.05).

TABLE 3
Effect of the drug to be tested on aortic plaques in ApoE−/− mice

| Group | Contrast | Model | Atorvastatin | AQ-C-a | DMS-ASD | DMS |
|---|---|---|---|---|---|---|
| Dose | — | — | 20 mg/kg | 300 mg/kg | 300 mg/kg | 20 mg/kg |
| % | 0.77 ± 0.26**** | 12.71 ± 1.88 | 6.86 ± 1.21** | 9.41 ± 1.02 | 7.82 ± 1.55** | 8.43 ± 0.95* |
| Patch | | | | | | |
| area | | | | | | |

### 3.5 Effect on Heart Weight Index of ApoE−/− Mice

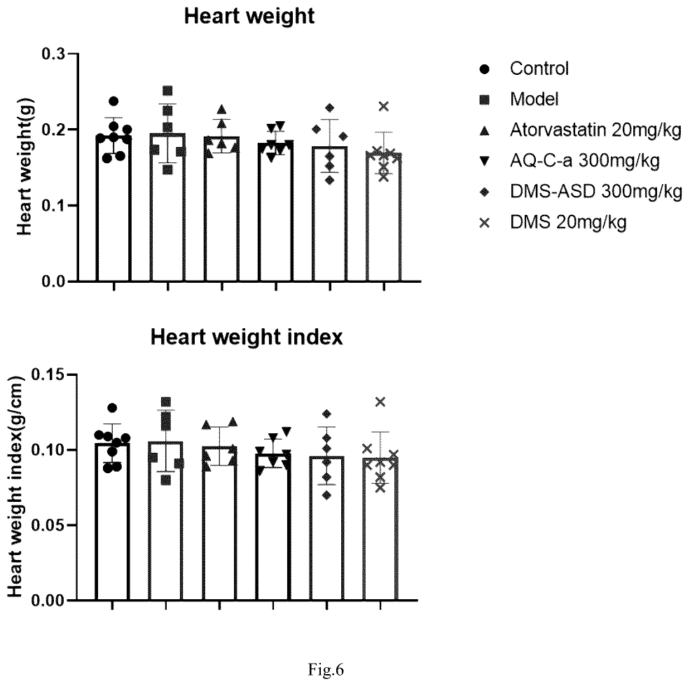

After euthanasia at the end of the experiment, the hearts of the animals were removed and weighed, and the weight was corrected using tibial length. The results are shown in Table 4 and FIG. 6: Compared with the control group, there was no significant difference in heart weight and heart weight index among the groups of mice (P>0.05).

TABLE 4
Effect of the tested drug on the heart weight index of ApoE−/− mice

| Group | Contrast | Model | Atorvastatin | AQ-C-a | DMS-ASD | DMS |
|---|---|---|---|---|---|---|
| Dose | — | — | 20 mg/kg | 300 mg/kg | 300 mg/kg | 20 mg/kg |
| Heart(g) | 0.192 ± 0.008 | 0.195 ± 0.016 | 0.192 ± 0.009 | 0.182 ± 0.006 | 0.178 ± 0.014 | 0.169 ± 0.010 |
| Shin | 1.838 ± 0.021 | 1.842 ± 0.024 | 1.875 ± 0.028 | 1.871 ± 0.029 | 1.858 ± 0.015 | 1.794 ± 0.020 |
| bone | | | | | | |
| (cm) | | | | | | |
| Cardiac | 0.105 ± 0.005 | 0.106 ± 0.008 | 0.102 ± 0.005 | 0.098 ± 0.004 | 0.096 ± 0.008 | 0.095 ± 0.006 |
| weight | | | | | | |
| index | | | | | | |
| (g/cm) | | | | | | |

### IV. Conclusion

Administering 300 mg/kg of DMS-ASD and 20 mg/kg of DMS for 10 consecutive weeks demonstrates multiple pharmacological effects, including inhibiting the increase in body weight, blood lipid TC, LDL-c, and plasma LDH in mice, as well as reducing the area of aortic atherosclerotic plaques.

The above is merely an embodiment of this application and is not intended to limit it. For those skilled in the art, various modifications and variations are possible. Any modifications, equivalent substitutions, improvements, etc., made within the spirit and principles of this application, should be deemed as falling within the scope of the claims of this application.

1. The method of dimyricetin-yl-diselenide in the preparation of a pharmaceutical composition, wherein the pharmaceutical composition is used for preventing, alleviating, and/or treating cardiovascular diseases.

2. The method according to claim 1, wherein the pharmaceutical composition is used for preventing and/or treating atherosclerosis.

3. The method according to claim 2, wherein the cardiovascular diseases are selected from the group consisting of aortic atherosclerosis, coronary atherosclerosis, coronary atherosclerotic heart disease, carotid and cerebral atherosclerosis, renal atherosclerosis, and atherosclerosis of the extremities.

4. A pharmaceutical composition for preventing, alleviating, and/or treating cardiovascular diseases, wherein the active ingredient is dimyricetin-yl-diselenide.

5. The pharmaceutical composition according to claim 4, wherein the pharmaceutical composition further contains a pharmaceutically acceptable carrier.

6. The pharmaceutical composition according to claim 4, wherein the pharmaceutical composition is used for preventing and/or treating atherosclerosis.
